(19) United States
(12) Patent Application Publication
(10) Pub. No.: US 2013/0310760 A1
(43) Pub. Date: Nov. 21, 2013

(54) Stopper Having a Scale Line Formed Thereon

(71) Applicant: Becton, Dickinson and Company, Franklin Lakes, NJ (US)

(72) Inventors: Milan Ivosevic, Kinnelon, NJ (US); Fred Burbank, Pompton Plains, NJ (US)

(21) Appl. No.: 13/897,749

(22) Filed: May 20, 2013

Related U.S. Application Data
(60) Provisional application No. 61/649,428, filed on May 21, 2012.

(51) Int. Cl. A61M 5/31

(52) U.S. Cl. CPC A61M 5/3129
USPC 604/207

(57) ABSTRACT

A stopper adapted for attachment with a plunger rod for use within a syringe barrel includes: a main body portion defining an open rearward end configured to receive the plunger rod and a closed front end; and a stopper scale line formed along an outer edge of the main body portion. The stopper scale line is formed to provide a visual enhancement of a portion of the stopper that aligns with volume measuring demarcations provided along the syringe barrel, and the stopper scale line contrasts with the main body portion of the stopper.

### CROSS-REFERENCE TO RELATED APPLICATION

This application claims the benefit of U. S. Provisional Patent Application No. 61/649,428 entitled “Stopper Having a Scale Line Formed Thereon” filed May 21, 2012, the entire disclosure of which is hereby incorporated by reference in its entirety.

### BACKGROUND OF THE INVENTION

1. Field of the Invention

The invention relates, in general, to a stopper assembly for use with a syringe and, more particularly, to a stopper assembly having a scale line formed thereon for improving the overall syringe scale line readability.

2. Description of Related Art

A typical syringe includes a barrel, a stopper positioned within the barrel, and a plunger rod coupled to the stopper. The barrel, usually made from plastic or glass, having a proximal end and distal end, acts as a reservoir for a fluid. The proximal end is open to receive the stopper and plunger rod. The distal end is connected to a cannula or adaptable to a hypodermic needle assembly to permit fluid to be drawn or expelled through the distal end. The stopper is attached to the plunger rod and provides a means of creating a liquid tight seal between the stopper and the syringe barrel so that movement of the plunger rod along the barrel causes liquid to be drawn into or expelled out of the syringe through the distal end. The stopper is moved through the syringe barrel by applying axial force to the plunger rod. In some instances, the stopper and the plunger rod may be integrally formed.

During use, the distal end of the syringe barrel is initially placed in contact with a fluid. For example, the needle on the syringe can be inserted into a liquid medication. As the plunger is retracted within the barrel, a process known as aspiration, a negative pressure is formed within the end of the barrel so as to cause the fluid to be drawn into the barrel. The syringe can then be moved to a second location where advancing the plunger within the barrel causes the fluid to be pushed or expressed out the discharge end of the barrel.

Syringes usually contain demarcations such as volume measuring indicia on the side of the barrel to indicate the volume of liquid contained within the syringe. The demarcations are usually black and are sometimes difficult to read during operation. In addition, among new syringe users and practitioners, there is an ever present doubt with respect to what part of the stopper needs to be aligned with the volume measuring indicia for an accurate medication dose.

Accordingly, a need exists for a user friendly syringe that intuitively defines which part of the stopper needs to be aligned with the volume measuring indicia provided on the syringe barrel for an accurate medication dose. In addition, a further need exists for a syringe that improves dose accuracy and scale readability even at reduced lighting conditions.

### SUMMARY OF THE INVENTION

It is an object of the invention to provide a user friendly syringe that intuitively defines which part of the stopper needs to be aligned with the volume measuring indicia provided on the syringe barrel for an accurate medication dose. A further object of the invention is to provide a syringe that improves dose accuracy and scale readability even at reduced lighting conditions.

The particularly disclosed stopper design includes a scale line formed thereon for improving the overall syringe volume measuring indicia readability.

According to one aspect of the invention, the stopper is adapted for attachment with a plunger rod for use within a syringe barrel. The stopper includes: a main body portion defining an open rearward end configured to receive the plunger rod and a closed front end; and a stopper scale line formed along an outer edge of the main body portion. The stopper scale line is formed to provide a visual enhancement of a portion of the stopper that aligns with volume measuring demarcations provided along the syringe barrel and contrasts with the remaining portions of the stopper.

The closed front end of the main body may be conically shaped with a tip. The stopper scale line may be formed along an outer edge of the main body portion at a base of the conically shaped closed front end.

The stopper may further include a perimetrical skirt extending around an outer circumference of the main body portion toward the closed front end of the main body portion. The stopper scale line may be formed along an upper edge of the perimetrical skirt.

A thickness of the stopper scale line may substantially be identical to a thickness of each of the volume measuring demarcations provided along the syringe barrel. The stopper scale line may have a color that is different than a color of the main body portion, thereby providing the opportunity to be used for color coding applications (e.g., different stopper color lines used on different syringe sizes).

The stopper scale line may be formed from a material that is different than the main body portion, and wherein the material forming the stopper scale line has a different color than the color of the material forming the main body portion. The stopper scale line may be formed from a different material than the main body portion. For instance, the stopper scale line may be formed from an elastic material that is overmolded onto a rigid material that forms the main body portion. Alternatively, the stopper scale line may be formed by laser inscription or may be printed onto the main body portion. In certain embodiments, the stopper scale line is formed by chemical etching.

According to another aspect of the present invention, a syringe is provided that includes: a) a substantially cylindrical syringe barrel having volume measuring demarcations extending along a length thereof and comprising a fluid dispensing end and an open end; b) a stopper configured to be received within the open end of the syringe barrel; and c) a plunger rod having a plunger rod body extending along a longitudinal axis, the plunger rod body comprising a front attachment end configured to engage the open rearward end of the main body portion of the stopper and a back end. The stopper includes: i) a main body portion defining an open rearward end configured to receive the plunger rod and a closed front end; and ii) a stopper scale line formed along an outer edge of the main body portion. The stopper scale line is formed to provide a visual enhancement of a portion of the stopper that aligns with the volume measuring demarcations provided along the syringe barrel as the stopper is moved through the syringe barrel and contrasts with the remaining portions of the stopper. the stopper scale line is formed to provide a visual enhancement of a portion of the stopper that aligns with volume measuring demarcations provided along the syringe barrel and contrasts with the remaining portions of the stopper

According to yet another aspect of the present invention, a plunger rod and stopper assembly adapted for use with a syringe barrel is provided. The assembly includes: a) a stopper configured to be received within the open end of the syringe barrel; and b) a plunger rod having a plunger rod body extending along a longitudinal axis, the plunger rod body comprising a front attachment end configured to engage the open rearward end of the main body portion of the stopper and a back end. The stopper includes: i) a main body portion defining an open rearward end configured to receive the plunger rod and a closed front end; and ii) a stopper scale line formed along an outer edge of the main body portion. The stopper scale line is formed to provide a visual enhancement of a portion of the stopper that aligns with volume measuring demarcations provided along the syringe barrel and contrasts with the remaining portions of the stopper. The stopper and the plunger rod may be separately or integrally formed.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is an exploded perspective view of a syringe barrel and plunger rod/stopper in accordance with an embodiment of the present invention.

FIG. 2 is a perspective view of a stopper in accordance with an embodiment of the present invention.

FIG. 3 is a partial cross-sectional perspective view of the stopper of FIG. 2 in accordance with an embodiment of the present invention.

FIG. 4 is front plan view of the stopper positioned within the syringe barrel in accordance with an embodiment of the present invention.

FIG. 5 is a perspective view of a stopper in accordance with an alternative embodiment of the present invention.

FIG. 6 is a partial cross-sectional perspective view of the stopper of FIG. 5 in accordance with an embodiment of the present invention.

### DESCRIPTION OF THE INVENTION

For purposes of the description hereinafter, the terms “upper”, “lower”, “right”, “left”, “vertical”, “horizontal”, “top”, “bottom”, “lateral”, “longitudinal”, and derivatives thereof, shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

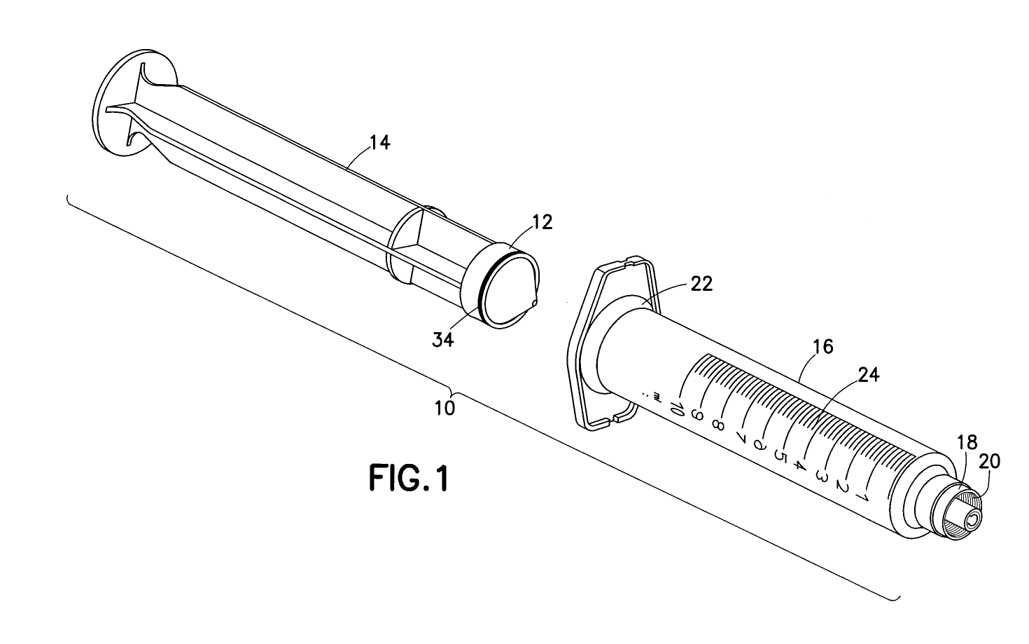

With reference to FIG. 1, a syringe, generally denoted as reference numeral 10, includes a stopper 12 and a plunger rod 14. The stopper 12 and plunger rod 14 are adapted for use within a syringe barrel 16. The stopper 12 and plunger rod 14 may be provided as separate components. In addition, it is also contemplated that the stopper features may be integrally formed with the plunger rod 14.

The syringe barrel 16 includes a distal or frontal end 18 which includes an outlet opening and/or a mechanism for attachment of a separate medical device (such as a catheter), shown in the form of a luer 20, and an open proximal or rearward end 22 for receiving the stopper 12 and plunger rod 14 assembly. However, while the use of a luer type attachment is illustrated in FIG. 1, this is not to be construed as limiting the present invention as any suitable type of attachment for syringe 10 has been contemplated. In addition, the syringe barrel 16 includes volume measuring demarcations 24 provided on a surface thereof by any conventional technique. The volume measuring demarcations 24 are provided to give the user an indication of the volume of fluid in the syringe barrel.

These volume measuring demarcations 24 are usually black and are sometimes difficult to read during operation. In addition, among new syringe users and practitioners, there is an ever present doubt with respect to what part of the stopper needs to be aligned with the volume measuring indicia for an accurate medication dose. The stopper of the present invention has been developed to address these issues.

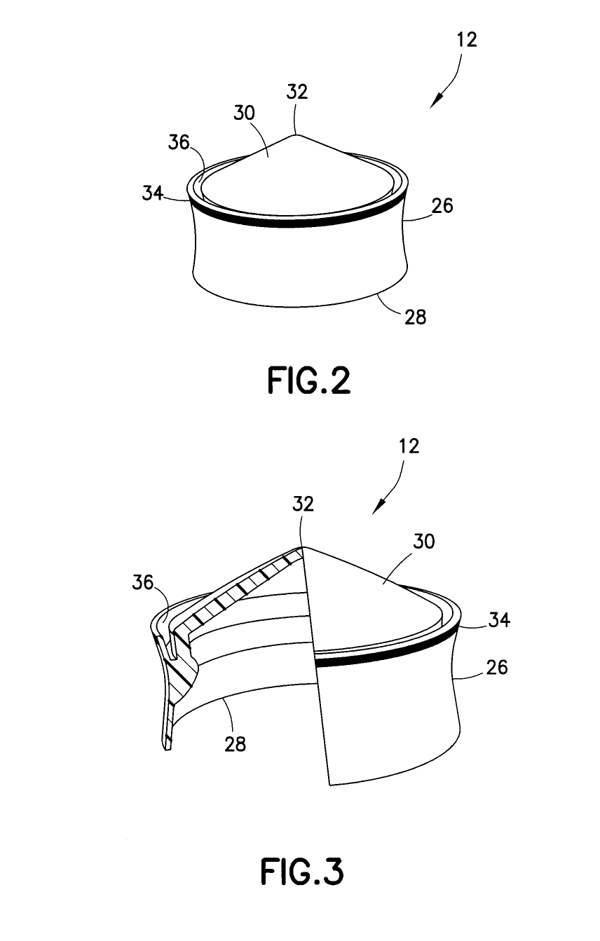

More particularly and with reference to FIGS. 2-4, the stopper 12 includes a main body portion 26 defining an open rearward end 28 configured to receive the plunger rod 14, and a closed front end 30. The closed front end 30 of the main body 26 is conically shaped with a tip 32. However, this shape of the closed front end 30 is not to be considered as limiting the present invention as the closed front end 30 may be flat or have any other suitable shape. A perimetrical skirt 36 is provided that extends around an outer circumference of the main body portion 26 toward the closed front end 30. The primary purpose of the perimetrical skirt 36 is to provide a seal between the main body portion 26 of the stopper 12 and an inner wall 38 of the syringe barrel 16.

A stopper scale line 34 is formed along an outer edge of the main body portion 26. More specifically, the stopper scale line 34 is formed at a base of the conically shaped closed front end 30 along an upper edge of the perimetrical skirt 36 as shown in FIGS. 2 and 3. The stopper scale line 34 aligns with the volume measuring demarcations 24 provided along the syringe barrel 16. More specifically, the stopper scale line 34 should be provided at the outer edge of the main body portion 26 of the stopper 12 that is required to be aligned with the volume measuring demarcations 24 in order to provide an accurate indication to a user of the volume of fluid remaining in the syringe barrel 16. Accordingly, the stopper scale line 34 intuitively defines which part of the stopper 12 needs to be aligned with the volume measuring demarcations 24 provided on the syringe barrel 16 for an accurate medication dose, thereby improving dose accuracy and the readability of the volume measuring demarcations 24.

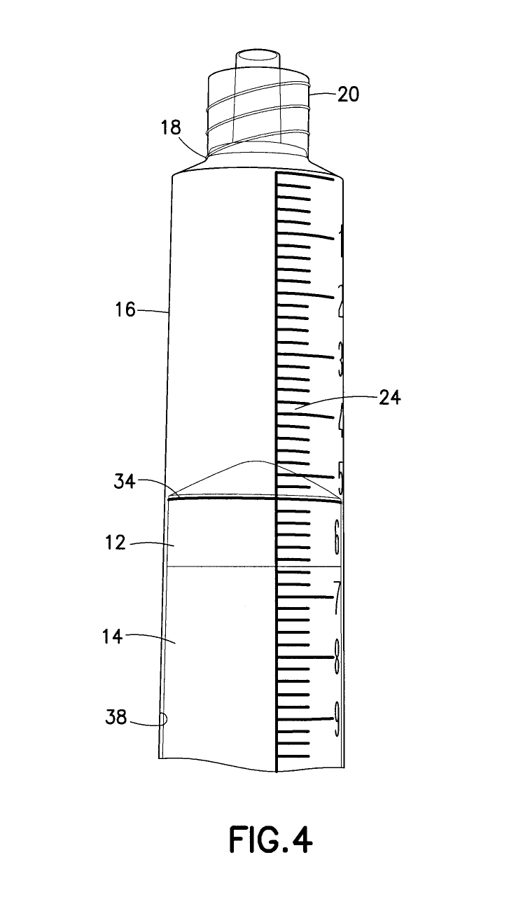

With specific reference to FIG. 4, a thickness of the stopper scale line 34 is identical or substantially identical to a thickness of each of the volume measuring demarcations 24 provided along the syringe barrel 16. This allows for the best performance and dose accuracy. Furthermore, the stopper scale line 34 must be positioned such that it is precisely aligned with the volume measuring demarcation indicating zero on the barrel, when the syringe is fully depressed. If the stopper scale line 34 is not positioned in this manner, then it cannot be used to provide an accurate indication of the volume of fluid in the syringe.

The stopper scale line 34 may be formed in a number of different ways. For instance, the stopper scale line 34 may be formed from an elastic material that is overmolded onto a rigid material that forms the main body portion 26. The elastic material used for the stopper scale line 34 may be selected from Thermoplastic Elastomers (TPE) such as Styrene Block Copolymers (TPS), Thermoplastic Polyolefins (TPO), Thermoplastic Vulcanisates (TPV), Thermoplastic Polyurethanes (TPU), Copolyesters, or other elastomers. The rigid material used to form the main body portion 26 may include, but is not limited to, thermoplastics such as polyolefines (e.g., PE, PP, and their copolymers), polyamides (e.g., nylons), polyesters (e.g., PET), polystyrene, polyurethane, polycarbonate, acrylonitrile-butadiene-styrene, fluoropolymers, ionomers, polyacrylates, or any other similar material. Any suitable molding process such as injection molding or compression molding may be used to overmold the stopper scale line 34 onto the main body portion 26. When the stopper scale line 34 is molded onto the main body portion 26, it provides a dual function. First, it provides elastic sealing with the inner wall 38 of the syringe barrel 16. In addition, the stopper scale line 34 is configured to be aligned with the volume measuring demarcations 24 on the syringe barrel 16 for improved dose accuracy. Furthermore, when the stopper scale line 34 and main body portion 26 are molded in this manner, the stopper scale line 34 may be provided as a color that is different than a color of the main body portion 26, thereby providing the opportunity to be used for color coding applications (e.g., different stopper color lines used on different syringe sizes).

Alternatively, the stopper scale line 34 may be formed by laser inscription. More specifically, a contrasting stopper scale line 34 can be inscribed on an outer edge of the main body portion 26 by a laser that would generate localized change of material properties, thereby providing a stopper scale line 34. The elastic material used for the main body portion on which the stopper scale line 34 is inscribed may be selected from Thermoplastic Elastomers (TPE) such as Styrene Block Copolymers (TPS), Thermoplastic Polyolefines (TPO), Thermoplastic Vulcanisates (TPV), Thermoplastic Polyurethanes (TPU), Copolyesters, or other elastomers and rubbers such as Polyisoprene. In addition, the use of laser inscription of the stopper scale line 34 improves the sliding behavior of the stopper 12 within the syringe barrel 16.

Syringes typically require slow and controlled initiation and maintenance of sliding movement of the stopper 12 over the inner wall 38 of the syringe barrel 16. It is well known that two stationary surfaces having a sliding relationship often exhibit sufficient resistance to initiation of movement that gradually increased force applied to one of the surfaces does not cause movement until a threshold force is reached, at which point a sudden sliding or shearing separation of the surfaces takes place. This sudden separation of stationary surfaces into a sliding relationship is herein referred to as “breakout” or “breakloose”.

“Breakout force” refers to the force required to overcome static friction between surfaces of a syringe assembly that has been previously moved in a sliding relationship, but has been stationary (“parked” or not moved) for a short period of time (for example, milliseconds to hours). A less well known but important frictional force is “breakloose force”, which refers to the force required to overcome static friction between surfaces of a syringe assembly that have not been previously moved in a sliding relationship or have been stationary for longer periods of time, often with chemical or material bonding or deformation of the surfaces due to age, sterilization, temperature cycling, or other processing.

The problems of excessive breakout and breakloose forces are related to friction. Friction is generally defined as the resisting force that arises when a surface of one substance slides, or tends to slide, over an adjoining surface of itself or another substance. Between surfaces of solids in contact, there may be two kinds of friction: (1) the resistance opposing the force required to start to move one surface over another, conventionally known as static friction; and (2) the resistance opposing the force required to move one surface over another at a variable, fixed, or predetermined speed, conventionally known as kinetic friction.

Depending on laser strength and wave length, material can be re-melted, slightly burned, molecules can be locally re-oriented, etc. These changes would result in localized change of elastic modulus of the stopper material at a front leading edge of the stopper 12, which plays an important role in sliding behavior of the stopper. The effects of this localized change of the elastic modulus of the stopper material at the front leading edge of the stopper 12 can reduce the force required to achieve breakout and/or breakloose forces, whereby transition of surfaces from stationary contact to sliding contact occurs without a sudden surge. When breakout or breakloose is complete and the leading edge of the stopper 12 and the inner wall 38 of the syringe barrel 16 are in sliding contact, they slide smoothly upon application of minimal force.

Still further, the stopper scale line 34 may be printed onto the main body portion 26 using any suitable known printing technology.

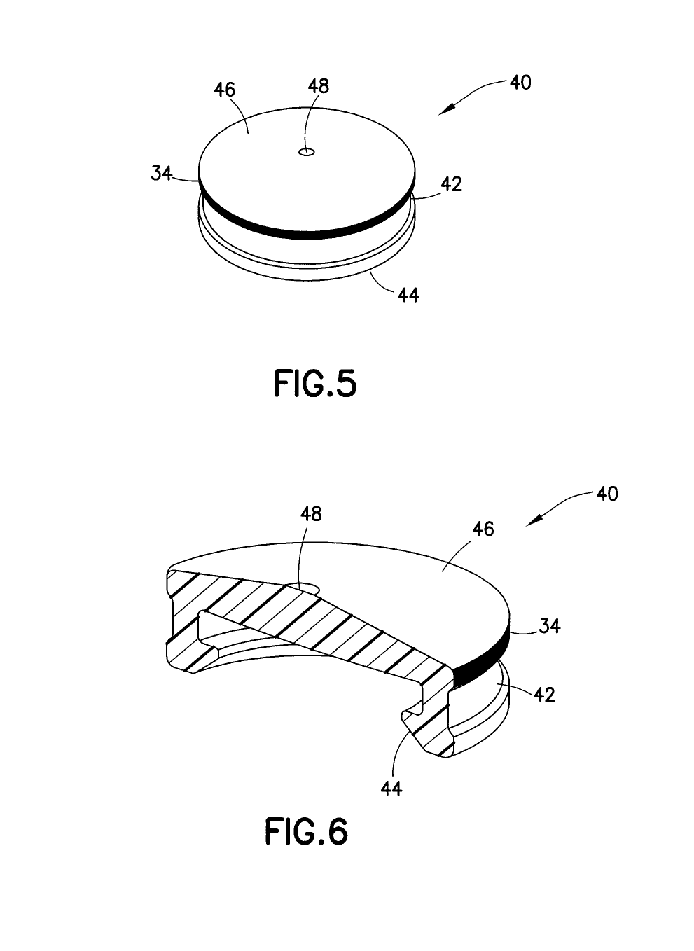

Although the stopper 12 has been discussed hereinabove as being of a configuration as shown in FIGS. 2 and 3, this is not to be construed as limiting the present invention as any stopper may be configured to utilize the stopper scale line 34. For instance, with reference to FIGS. 5 and 6, a stopper, generally denoted as reference numeral 40, includes a main body portion 42 defining an open rearward end 44 configured to receive the plunger rod 14, and a closed front end 46. The closed front end 46 of the main body 42 is conically shaped with a tip 48. However, this shape of the closed front end 46 is not to be considered as limiting the present invention as the closed front end 46 may be flat or have any other suitable shape.

A stopper scale line 34 is formed along an outer edge of the main body portion 42. More specifically, the stopper scale line 34 is formed at a base of the conically shaped closed front end 46 as shown in FIGS. 5 and 6. The stopper scale line 34 aligns with the volume measuring demarcations 24 provided along the syringe barrel 16 as discussed in greater detail hereinabove.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of invention which is to be given the full breadth of the claims appended and any and all equivalents thereof.

The invention claimed is:

1. A stopper adapted for attachment with a plunger rod for use within a syringe barrel, the stopper comprising:
a main body portion defining an open rearward end configured to receive the plunger rod and a closed front end; and
a stopper scale line formed along an outer edge of the main body portion,
wherein the stopper scale line provides a visual enhancement portion of the stopper that aligns with volume measuring demarcations provided along the syringe barrel, and
wherein the stopper scale line contrasts with the main body portion of the stopper.

2. The stopper of claim 1, wherein the closed front end of the main body portion is conically shaped with a tip.

3. The stopper of claim 2, wherein the stopper scale line is formed along the outer edge of the main body portion at a base of the conically shaped closed front end.

4. The stopper of claim 1, further comprising a perimetrical skirt extending around an outer circumference of the main body portion toward the closed front end of the main body portion.

5. The stopper of claim 4, wherein the stopper scale line is formed along an upper edge of the perimetrical skirt.

6. The stopper of claim 1, wherein a thickness of the stopper scale line is substantially identical to a thickness of each of the volume measuring demarcations provided along the syringe barrel.

7. The stopper of claim 1, wherein the stopper scale line has a color that is different than a color of the main body portion.

8. The stopper of claim 1, wherein the stopper scale line is formed from a different material than the main body portion.

9. The stopper of claim 1, wherein the stopper scale line is formed from a material that is different than the main body portion, and wherein the material forming the stopper scale line has a different color than the color of the material forming the main body portion.

10. The stopper of claim 1, wherein the stopper scale line is formed from an elastic material that is co-molded or overmolded onto a rigid material that forms the main body portion.

11. The stopper of claim 1, wherein the stopper scale line is formed by laser inscription.

12. The stopper of claim 1, wherein the stopper scale line is formed by chemical etching.

13. The stopper of claim 1, wherein the stopper scale line is printed onto the main body portion.

14. The stopper of claim 1, wherein the stopper scale line is attached to the main body portion by at least one of welding and gluing.

15. The stopper of claim 14, wherein the stopper scale line has a different color than the color of the material forming the main body portion.

16. A syringe comprising:
a) a substantially cylindrical syringe barrel having volume measuring demarcations extending along a length thereof and comprising a fluid dispensing end and an open end;
b) a stopper configured to be received within the open end of the syringe barrel, the stopper comprising:
i) a main body portion defining an open rearward end and a closed front end; and
ii) a stopper scale line formed along an outer edge of the main body portion; and
c) a plunger rod having a plunger rod body extending along a longitudinal axis, the plunger rod body comprising a front attachment end configured to engage the open rearward end of the main body portion of the stopper and a back end,
wherein the stopper scale line provides a visual enhancement portion of the stopper that aligns with the volume measuring demarcations provided along the syringe barrel as the stopper is moved through the syringe barrel, and
wherein the stopper scale line contrasts with the main body portion of the stopper.

17. The syringe of claim 16, wherein the closed front end of the main body portion of the stopper is conically shaped with a tip.

18. The syringe of claim 17, wherein the stopper scale line is formed along the outer edge of the main body portion at a base of the conically shaped closed front end.

19. The syringe of claim 16, wherein the stopper further comprises a perimetrical skirt extending around an outer circumference of the main body portion toward the closed front end of the main body portion.

20. The syringe of claim 19, wherein the stopper scale line is formed along an upper edge of the perimetrical skirt.

21. The syringe of claim 16, wherein a thickness of the stopper scale line is substantially identical to a thickness of each of the volume measuring demarcations provided along the syringe barrel.

22. The syringe of claim 16, wherein the stopper scale line has a color that is different than a color of the main body portion.

23. The syringe of claim 16, wherein the stopper scale line is formed from a different material than the main body portion.

24. The syringe of claim 23, wherein the stopper scale line is formed from an elastic material that is overmolded onto a rigid material that forms the main body portion.

25. The syringe of claim 16, wherein the stopper scale line is formed by laser inscription.

26. The syringe of claim 16, wherein the stopper scale line is printed onto the main body portion.

27. A plunger rod and stopper assembly adapted for use with a syringe barrel, the assembly comprising:
a) a stopper configured to be received within an open end of the syringe barrel, the stopper comprising:
i) a main body portion defining an open rearward end and a closed front end; and
ii) a stopper scale line fanned along an outer edge of the main body portion; and
b) a plunger rod having a plunger rod body extending along a longitudinal axis, the plunger rod body comprising a front attachment end configured to engage the open rearward end of the main body portion of the stopper and a back end,
wherein the stopper scale line provides a visual enhancement portion of the stopper that aligns with volume measuring demarcations provided along the syringe barrel, and
wherein the stopper scale line contrasts with the main body portion of the stopper.

28. The assembly of claim 27, wherein the stopper and the plunger rod are integrally formed.

29. The assembly of claim 27, wherein the stopper further comprises a perimetrical skirt extending around an outer circumference of the main body portion toward the closed front end of the main body portion.

30. The assembly of claim 29, wherein the stopper scale line is formed along an upper edge of the perimetrical skirt.

31. The assembly of claim 27, wherein a thickness of the stopper scale line is substantially identical to a thickness of each of the volume measuring demarcations provided along the syringe barrel.

32. The assembly of claim 27, wherein the stopper scale line has a color that is different than a color of the main body portion.

33. The assembly of claim 27, wherein the stopper scale line is formed from a different material than the main body portion.

34. The assembly of claim 27, wherein the stopper scale line is formed from an elastic material that is overmolded onto a rigid material that forms the main body portion.

35. The assembly of claim 27, wherein the stopper scale line is formed by laser inscription.

36. The assembly of claim 27, wherein the stopper scale line is printed onto the main body portion.
